(19) United States
(12) Patent Application Publication
(10) Pub. No.: US 2023/0418304 A1
(43) Pub. Date: Dec. 28, 2023

(54) MOBILE INDUSTRIAL ROBOTIC PLATFORM THAT CAN BE USED FOR DIFFERENT PURPOSES THROUGH MODULES THAT CAN BE CHANGED BY THE USER

(71) Applicant: Ara Robotik Ve Yapay Zeka Teknolojileri Anon Sirk, Yenimahalle/Ankara (TR)

(72) Inventor: Kemal Eren SERBETCI, Çankaya/Ankara (TR)

(21) Appl. No.: 18/250,503

(22) PCT Filed: Jun. 10, 2021
(86) PCT No.: PCT/TR2021/050566

§ 371 Date: Apr. 25, 2023

(30) Foreign Application Priority Data
Oct. 28, 2020 (TR) TR 2020/17227

(51) Int. Cl. G05D 1/02; A47L 7/00; A47L 9/02; A47L 9/14; A47L 9/24; A47L 9/28; A47L 11/16; A47L 11/18; A47L 11/20; A47L 11/30; A47L 11/40; A61L 2/24; A61L 2/14; A61L 2/22; A61L 2/10

(52) U.S. Cl. CPC G05D 1/0246; G05D 1/0257; A47L 7/0038; A47L 7/0061; A47L 7/0085; A47L 9/02; A47L 9/14; A47L 9/24; A47L 9/2805; A47L 9/2873; A47L 9/2884; A47L 11/161; A47L 11/185; A47L 11/201; A47L 11/305; A47L 11/4005; A47L 11/4011; A47L 11/4016; A47L 11/4025; A47L 11/4038; A47L 11/4041; A47L 11/4044; A47L 11/405; A47L 11/4083; A47L 11/4088; A61L 2/24; A61L 2/14; A61L 2/22; A61L 2/10; A01D 34/008

(57) ABSTRACT

The invention is related to a mobile industrial autonomous device that can be used to perform different functions with the modules (8) of different features which can be easily changed by the user on the same platform. Although the main function targeted with the invention is the cleaning of indoor spaces, functions such as polishing, mowing, fertilizing, spraying etc. can also be provided by the robotic platform of the invention with modules that can be easily changed by the user. It will move automatically by the help of the lidar unit (4) and the camera units on the autonomous device, scan and map the environment and determine a route for itself accordingly and perform its function.

### TECHNICAL FIELD

The invention relates to mobile industrial autonomous device that can be used to perform different functions through modules with very different features that can easily be changed by the user on the same platform. Although the main function targeted with the invention is the cleaning of indoor spaces, functions such as polishing, mowing, fertilizing, spraying etc. can also be provided by the robotic platform of the invention with modules that can be easily changed by the user. The autonomous device will move automatically with the lidar and camera units thereon, scan and map the environment, determine a route accordingly and fulfil its function. This map stored in its memory can be changed later by the user or by obstacles/spatial changes. The device will be able to identify and record these obstacles/changes with different sensors on it.

### STATE OF THE ART

The maintenance of indoor/outdoor spaces and gardens around the world is generally done with large, heavy and in any way single-function devices usually driven behind and are used and/or monitored by the user. In other words, a single device is used for cleaning/maintaining carpet floors, and then a different devices must be purchased and used for cleaning hard floors or mowing.

When it comes to commercial/industrial space sizes, the sizes, weights and prices of these devices are too high considering they have only one function.

It is also a common problem for the functions to be performed incompletely, incorrectly or problematically in cleaning/polishing/mowing/spraying devices lead by a human due to reasons such as forgetting, distraction, interference of other tasks and etc.

The cleaning of the functional mechanisms of the devices used in different functions in different places is also done by the user. This is a constant obstacle for the device to be fully autonomous as it still requires human intervention in any case.

In fact, even though many different robotic mechanisms are used for domestic purposes within the current technique, the use of autonomous or semi-autonomous robotic mechanisms for the cleaning function of industrial areas is very limited. Although the mentioned mechanisms are used limitedly in real life, they become the subject of patent applications.

Patent application numbered EP2752726 describes an autonomously operable surface cleaning machine and a method for said surface cleaning. At least one embodiment of the invention generally refers to an automatically operable surface treatment machine and/or a method for surface treatment and/or a computer program that allows operating the computer controlled surface treatment machine.

According to the description of the application numbered EP2752726, the machine of the application can also be operated automatically. However, the machine of the application can be used for only one single cleaning function. For example, if users want wet cleaning on the floor surface, they will have to operate one machine, and if they want to vacuum the floor (dry cleaning), they will have to use a separate machine. In this case, the user will have to buy and keep all the machines with different functions. This situation will exhaust the user in terms of both investment costs and operating costs.

When the autonomous device patent studies carried out to date are examined, it is thought that different applications for a single function are known, but the structures that provide full automation in all of the charging, liquid filling and discharge, dust container discharge, self-cleaning, and orientation issues, and that contain modules that can be changed by the user are not used within the current technique.

### Problems That the Invention Aims To Solve

The aim of the invention is to create an autonomous robotic mechanism in which different functions can be provided by attaching and removing different modules in order to implement different functions.

The preferred embodiment of the invention describes the autonomous robotic mechanism that is suitable for mounting-demounting of different modules to clean industrial areas.

Within the scope of different embodiments of the invention, the system can be converted into a device to be used for polishing, mowing, fertilizing, spraying, surface disinfection, etc. by adding different modules to the robotic mechanism.

The user can convert the system she/he already purchased by removing the cleaning modules and adding other modules instead used for only functions like mowing, fertilizing etc. to perform these functions.

Similarly, the user can remove the dry cleaning module used for floor sweeping and add the wet cleaning module to the system, and thereby allow the robotic mechanism to provide different functions.

It is also possible for different modules to be added to or removed from the robotic mechanism of the invention without needing the user to change the modules.

For example, in the case the system is programmed to perform indoor cleaning, in line with the previously entered system information, the robotic mechanism will first come out of the docking station equipped with a sweeping module. After the sweeping is completed, the robotic mechanism will return to the docking station, and with the instruction of the system, it will leave the sweeping unit and can be equipped with a wet cleaning unit.

The robotic mechanism will be able to be operated autonomously, in a way that it can perform the necessary different functions on its own without human assistance, in line with the previously defined program.

In this way, the robotic mechanism of the invention can be operated in the area after directed to there, without needing the presence of a human in that area. For example, in the case the necessary program is defined for the robotic mechanism, the robotic mechanism will be able to perform all the necessary cleaning works at night using different modules.

By connecting different modules to the same robotic mechanism body and performing different tasks, costs both for the investment on the cleaning staff and the operation are reduced.

The autonomous operation of the entire system, including the replacement of modules of the robotic mechanism, will allow an additional cost saving as it will save manpower.

The fact that works such as cleaning etc. can be carried out independently of people will both reduce the margin of the human error and eliminate human-based security handicaps that may arise.

### DESCRIPTION OF DRAWINGS

FIG. 1. Exploded view of the autonomous robotic mechanism

FIG. 2. Detailed view of the sensor on the front part belonging to the autonomous robotic mechanism

FIG. 3. Detailed view of the sensors on the joint body with the autonomous robotic mechanism

FIG. 4. Side view of the autonomous robotic mechanism

FIG. 5. Side view of the autonomous robotic mechanism with the manual cleaning module is demounted and dry-cleaning module is mounted

FIG. 6. Side view of the robotic mechanism with wet cleaning modules and polishing module are demounted

FIG. 7. Side view of the robotic mechanism together with filling station

FIG. 8. Side view of the robotic mechanism with UVC disinfection module is demounted

FIG. 9. Side view of the robotic mechanism with ULV disinfection module and ULV tank demounted

### DESCRIPTION OF THE REFERENCES IN FIGURES

A. Filling station
1. Joint main body
2. Power supply
3. Joint electronic control and hardware unit
4. Lidar unit
5. Detector
   5.1. IR Impact sensor
   5.2. US Impact sensor
   5.3. TOF Sensor
   5.4. IR Fall sensor
   5.5. IR Wall tracking sensor
6. Bumper
7. Filling and discharge units
   7.1. Dirty water tank
8. Module
   8.1. Dry cleaning module
   8.2. Manual cleaning module
   8.3. Disc brush washing module
   8.4. Cylinder brush washing module
   8.5. Polishing module
   8.6. UVC disinfection module
   8.7. ULV disinfection module
9. Module slot
10. Module power input-output
11. Air suction connection
12. Module fluid inlet
13. Liquid suction connection
14. Robotic Washing unit
15. Charging unit
16. Liquid filling inlet
17. Top connection port
18. ULV tank

### DESCRIPTION OF THE INVENTION

The invention is a mobile industrial autonomous device that can be used to perform different functions with modules (8) with very different features that can be easily changed by the user on a single joint platform.

It has a software to do its own mapping and to navigate based on this map. By means of this navigation software, the user can name the parts of the place and direct the device only to them, or mark the places on the map on the screen where the device should stay away.

The functions to be performed by the device are determined through modules (8) that can be changed by the user easily and in a very short time. These modules (8) are automatically recognized by the device and the relevant software is activated.

When it is done with whatever function it performs, the device will return to the filling base (A) (charge/filling/cleaning unit) designed to perform joint operation, where it will self-clean and fill to prepare itself for the new duty cycle.

For the autonomous device of the invention to provide different functions, modules with different functions can be added or removed from the device. By this way, devices with different functions can be created by demounting and mounting different modules (8) on the same joint main body (1).

According to the preferred embodiment of the invention, the modules (8) are connected to the device by humans. However, in different embodiments of the invention, the modules (8) can mounted-demounted to the device by the system autonomously without needing human interference.

The device of the invention is constructed on a joint main body (1). The joint main body (1) is equipped with module slot (9) for different modules (8) to be mounted-demounted on it.

The joint main body (1) is also associated to at least one power supply (2) and joint electronic control and hardware unit (3).

In the preferred embodiment of the invention, the power supply (2) is in the form of a rechargeable battery.

For the device to map the environment, the joint main body (1) is equipped with lidar (4) unit.

Similarly, for the device to move autonomously, the joint main body (1) has various detectors (5) on it. According to the preferred embodiment of the invention, the detectors (5) are in the form of sensors.

To be able to control the different variables belonging to the environment the device will be used, the joint main body can be operated by positioning different types of detectors (5) to different parts in different numbers.

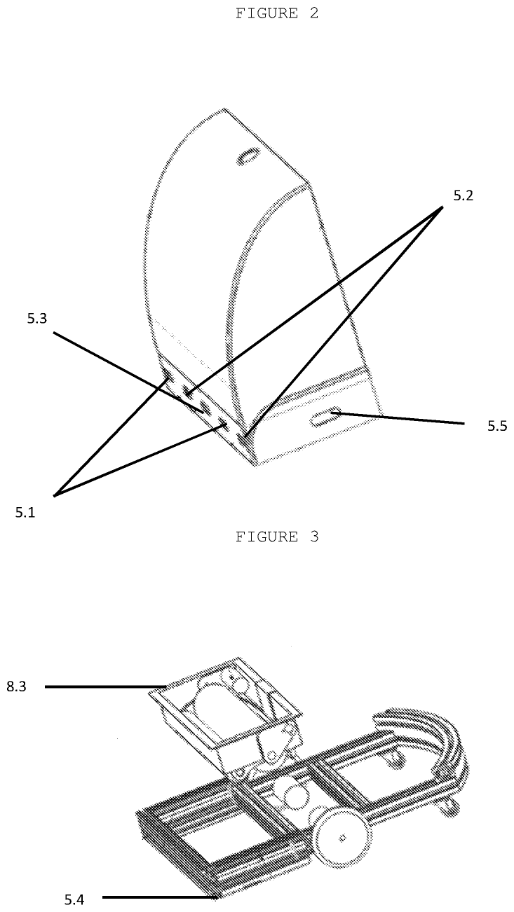

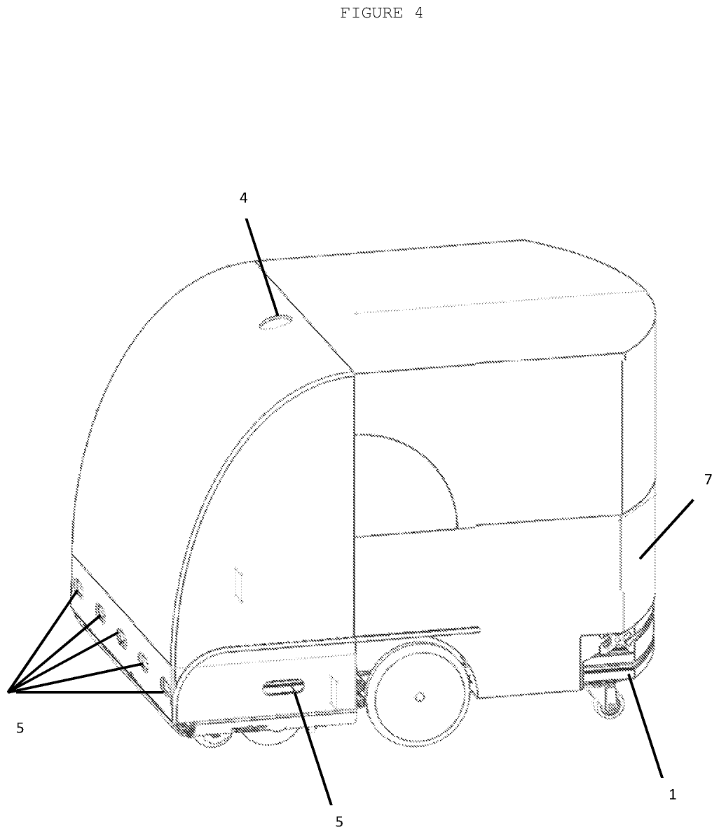

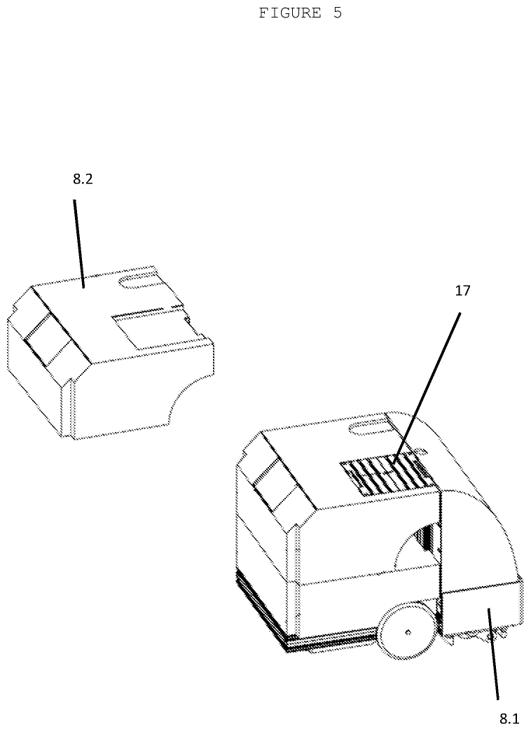

According to FIGS. 2 and 3, the device is equipped with IR impact sensor (5.1), US impact sensor (5.2), TOF sensor (5.3), IR fall sensor (5.4) and IR wall tracking sensor (5.5).

To detect the potential impacts during the movement of the device, the joint main body (1) is equipped with a bumper (6) associated to micro switches. The impact created on the bumper by the impact of the device will activate the micro switches and send a signal to the joint electronic control and hardware unit (3) for the device to be stopped instantly.

Modules (8) are associated with filling and discharge units (7) so that liquid can be transferred to them. In this way, when a module (8) that creates a fluid requirement is connected to the device, the fluid requirement of the module (8) will be met.

In case the module (8) needs liquid, the module can have a liquid inlet (12) in the module structure.

In order to withdraw the liquid used by the module, the device can also be equipped with a liquid suction connection (13).

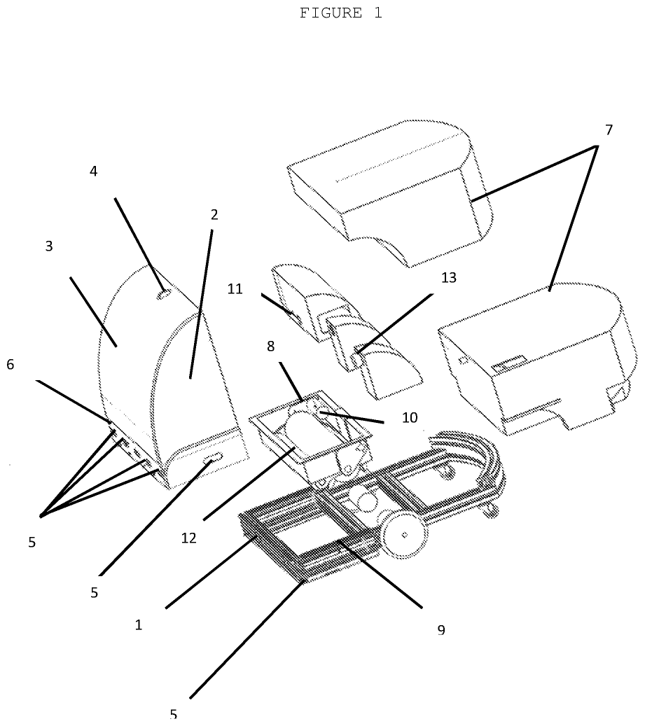

According to FIG. 1, the energy needed in the module (8) can be supplied by the module energy input-output (10).

In case the module (8) needs vacuum, the vacuum will be provided by the air suction connection (11).

By taking into account the predetermined time and the unit mounted thereon by the user (B), the device will leave the filling station (A) by driving the wheel motors with the power it takes from the power supply (2) and in line with the instruction coming from the joint electronic control and hardware unit (3) and will immediately start mapping by means of lidar unit (4). If the user has pinned a direction, obstruction or route on the predetermined map, the device will start moving by taking this into account. Otherwise, the map is reconstructed by the device.

This map will be stored within the memory of the device and be ready for the next use, but the device will automatically update the map checking the potential obstructions and changes in each operation. The device detects the environment by the help of lidar unit (4) and the detectors (5).

As people and other animates may be present in the operation environment of the device during its operation who can prevent the device from moving, to perform instantaneous obstruction identification and prevention, IR impact sensor (5.1), US impact sensor (5.2), TOF sensor (5.3), IR fall sensor (5.4) and IR wall tracking sensor (5.5) will always be on and continuously feed the main hardware with data. There must be at least 5 (five) of these detectors in different elevations on the front of the device to prevent any voids in the sight of the device, at least 1 (one) on the side of the device for wall tracking, at least 2 (two) on the back of the device to be used in approaching the filling station (A), and at least 4 (four) on the bottom of the device to be used in fall prevention. As the device will be used in commercial and industrial environment, the lidar unit composing the base of the navigation system shall have 16-20 m range. When the device encounters any obstacle, it will slow down, process the information coming from the sensors (5) and the lidar unit (4) in the joint electronic control and hardware unit (3) and decide in which direction and at what speed to move. The device performs these movements completely automatically.

Despite all this sensor (5) information, if any obstacle cannot be seen or processed quickly enough, the micro switches in the bumper (6) unit on the front will be activated when the device hits something or something hits it and send the joint electronic control and hardware unit (3) a signal to immediately stop the device. The joint electronic control and hardware unit (3) will then stop the device by cutting the power to the driving wheels and the attached processing unit. After a short waiting period, the device will control its entire environment without moving by means of the sensors (5) and the lidar unit (4), and if there is a safe movement area, it will continue its operation.

The general navigation system of the device is solved with SLAM (Simultaneous Localization And Mapping) and ROS (Robot Operating System) software structures, which are currently available as open source. By using these two software, it is possible for the device to do instant mapping, and to process and mark this map both autonomously and with user support. With the system we developed, it is possible to mark the sections on the map, which consists of points read by the lidar unit (4) with the TOF (Time Of Flight) method, to name these sections, and to mark the places we want or do not want the device to enter. The device will be able to be sent to one or more named areas when desired to perform its function. This map, which will be stored digitally within the device, can be shared with other robots in the environment with IOT algorithms when necessary, and division of labour can be ensured. In this way, it will be possible to complete works with cooperation that cannot be practically handled by a single device.

The device cannot travel at a ground height difference of more than 25 mm, so the IR fall sensors (5.4) were programmed to detect elevations and low areas on the device's path.

Right after the first map is obtained, it starts to function according to the commands it receives from the joint electronic control and hardware unit (3) and a route it creates. This function can be dry or wet cleaning, mowing, UV disinfection, disinfection, etc., depending on the installed module (8).

As soon as the device starts its first movement, all sensors (5) are activated and they start to measure continuously. At the same time, the micro switches connected to the bumper (6) are energized and they are kept on alert to stop the device against an unpredictable crash.

When the forward movement starts according to the map created and the route determined, the function of the unit loaded thereon by the user begins to be performed.

Replacing the modules of the device of the invention by humans is performed as follows.

The user opens the power supply (2) section (cup) on the front of the device in a circular manner to the front and exposes the unit to be replaced. During this opening, the device automatically cuts off the energy coming from the power supply (2) and prevents any malfunctioning. The locking structure common to each module (8) is released with a total of 6 (six) manually removable clamps. The released module (8) is removed by the user and the different module (8) is inserted. The locking clamps are secured and the cover is closed and secured again with a circular motion. When the energy from the power supply (2) is given again, the joint electronic control and hardware unit (3) automatically recognizes the added module (8) and directs itself to the relevant software.

The user then adds one of the replaceable supply modules (8) to its place. The joint electronic control and hardware unit (3) automatically recognizes the added module (8) and prepares itself for operation. The device is ready for autonomous operation after this stage.

When the robotic mechanism performs the dry cleaning function, the dry cleaning module (8.1) can be connected to the device. The dry cleaning module (8.1) dandy brush will start pounding and fluffing the floor, and the main brush and flexible oscillating side brushes will pick up and remove the released dirt and dust. In the meantime, dirt and dust collected by the brushes will be filled into the container with the vacuum produced by the fan in its unit that began to work with the command it received from the joint electronic control and hardware unit (3).

The fullness of the dust container is detected by the fullness sensor in the unit and transmitted to the joint electronic control and hardware unit (3).

The dry cleaning ends when the dust container in the unit is full, the power supply (2) is low, or the mapped space is completely treated, and the device returns to the filling station (A).

According to FIG. 1, the dust container is structured within or associated with the filling and discharge units (7).

According to one of the preferred embodiments of the invention, the device can be operated autonomously as well as manually. In this case, the device may contain a manual cleaning module (8.2).

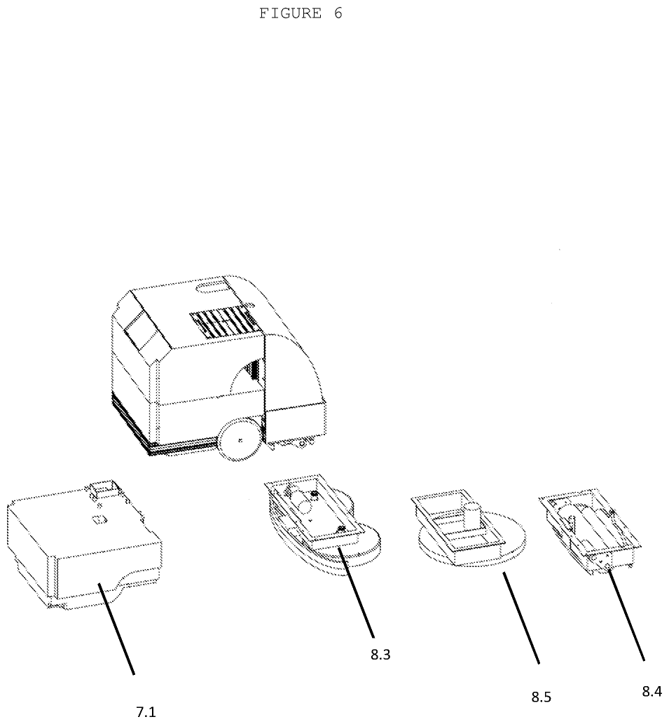

According to FIG. 5, the manual cleaning module (8.2) can also contain a hose and cleaning noses in an additional box in the upper empty part of the device.

If desired, the user will be able to perform manual dry cleaning by using the manual cleaning module (8.2), removing the hose, attaching it to the special connection on the container and operating the vacuum, when the user goes to the area to be cleaned.

When this hose is attached, the air inlet from the existing module is automatically closed to prevent pressure loss.

If the robotic mechanism is in the wet cleaning function mode, the front dry vacuum unit in the wet cleaning unit will start absorbing the dust and dirt to the container with the command it receives from the joint electronic control and hardware unit (3), then the liquid spray nozzles will spray the detergent-added water to the floor under pressure, the main brush and the flexible oscillating side brushes will foam the detergent liquid on the floor and remove the dirt. The dirty liquid accumulating in front of the rearmost wiper will be sucked into the dirty water tank by the fan in the unit. The dirty water accumulated in the dirty water tank will be renewed after passing through a series of filters and transferred to the clean tank, thus increasing the cleaning life of the device with a single tank of clean water.

The fullness of the dirty water tank (7.1) is detected by the fullness sensor in the unit and transmitted to the joint electronic control and hardware unit (3). Likewise, when the clean water supply ends it is detected and transmitted to the joint electronic control and hardware unit (3).

The wet cleaning process ends when the liquid supply to the unit is terminated, the dirty water tank is full, the power supply (2) is low, or the mapped space is completely treated, and the device returns to the filling station (A).

Structures such as dirty water tank (7.1), clean water tank etc. described in this process are formed within or associated with the filling and discharge units (7).

FIG. 6 shows the robotic mechanism and the wet cleaning process units that can be connected to it. Disc brush washing module (8.3) and cylinder brush washing module (8.4) can be given as examples of these units.

FIG. 6 also shows a polishing module (8.5) that can be added to the robotic mechanism. The polishing module (8.5) aims to provide the polishing function of the robotic mechanism.

According to the preferred application of the invention, the single rotating brush in the structure of the polishing module (8.5) will start to rotate with the command it receives from the joint electronic control and hardware unit (3), and the polishing will begin by the polish sent by the dosage unit beginning to flow with the command it receives from the joint electronic control and hardware unit (3). During the polishing operation, the fullness of the polish supply tank is continuously detected by the filling sensor and transmitted to the joint electronic control and hardware unit (3).

The polishing operation ends when the polish supply ends, the power supply (2) is low, or the mapped space is completely treated, and the device returns to the filling station (A).

Storage members such as polish supply tank etc. are structured within or in association with the filling and discharging units (7).

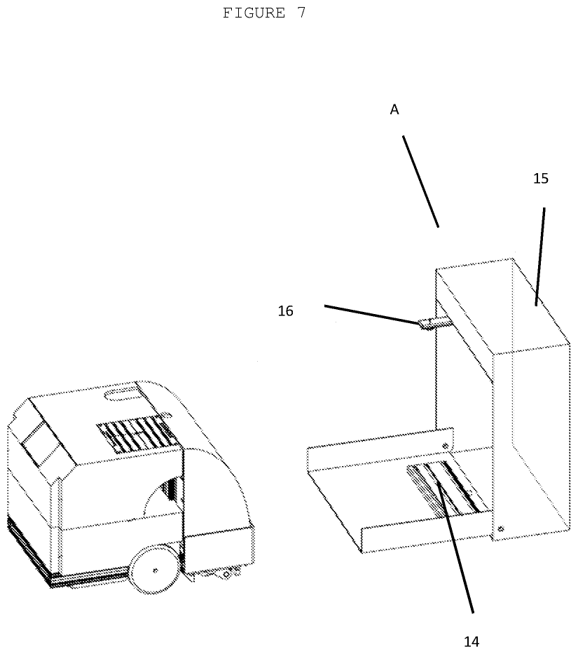

FIG. 7 shows the filling station (A) and the robotic mechanism together.

According to FIG. 7, the filling station (A) contains the robotic washing unit (14) within itself. The robotic washing unit (14) aims to wash and clean the modules attached to the robotic mechanism after their operations. For example, the cleaning of the disk brush washing module (8.3) and the cylinder brush washing module (8.4) can be provided by the robotic washing unit (14).

When the robotic mechanism approaches the filling station (A), it can also be cleaned by washing the bottom of the device with pressurized water. This process can also be provided by the robotic washing unit (14).

According to the preferred application of the invention, the robotic washing unit (14) placed in an additional horizontal platform belonging to the filling station (A) will be able to wash and clean the bottom of the device with the water sprayed through the nozzles by making use of the pressure of the normal tap water.

The dirty water collected at this time will be drained by the pool structure formed within the filling station (A). Also, the robotic washing unit (14) can be equipped with a moving comb system to clean the dirt accumulated on the brushes.

Again, according to FIG. 7, the filling station (A) also contains the filling unit (15). When the robotic mechanism approaches the filling station (A), its power supply/s (2) can be charged by the charging unit (15).

According to FIG. 7, the filling station (A) is equipped with a liquid filling inlet (16). Thus, when the robotic mechanism approaches the filling station (A), it can receive the necessary liquid supply by using the liquid filling inlet (16).

According to one of the preferred applications of the invention, disinfection of the application environment can be achieved by using the cleaning function HOCl. HOCl is obtained by electrolysis of a mixture of water and salt within the device.

In order for the HOCl system to be operated, the device can be designed to include the connections of the modular unit to be used in cleaning and preheat tank connections to be used in hot water or steam cleaning.

If the system will use hot water for this purpose or for other purposes, the tank and the accessories required for transferring the water to be heated here to the upper tank of the robot on which the wet module is mounted can also be added.

In addition to hot water, the device will also be able to contain structures that allow cleaning with steam. Steam can also be provided by the hot water module described above.

In case the steam generation unit is added externally, the tank and the necessary accessories for transferring the water to be preheated here to the upper tank of the robot equipped with the steam cleaning module, metal steam boiler, 24V water boiling apparatus, steam control valves, steam nozzles and special plush or micro fibre coated steam are connected to the device.

According to one of the preferred embodiments of the invention, the HOCl preparation unit and the hot water/steam preparation unit are associated with the filling station (A). Thus, the robotic mechanism can take the HOCl and hot water/steam prepared in filling station (A) readily.

If the robotic mechanism is in the spraying mode, the spraying unit will spread and spray the drug mixture coming under pressure from the filling and discharge units (7), which act as the spraying tank, and the connected dosing pump, to the floor via nozzles, with the command it receives from the joint electronic control and hardware unit (3). The fullness of the spraying tank is detected by the filling sensor associated with the filling and discharge units (7) and transmitted to the electronic hardware unit.

The spraying process ends when the disinfectant supply ends, the power supply (2) is low, or the mapped location is completely treated, and the device returns to the filling station.

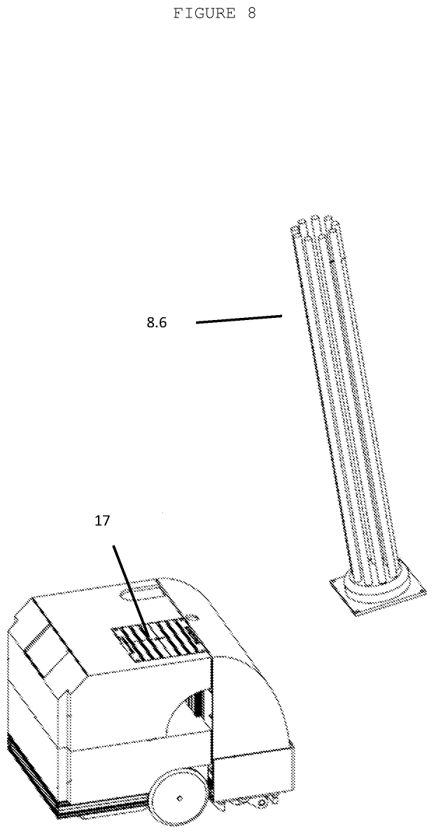

In the UV disinfection mode of the robotic mechanism, a UVC disinfection module (8.6) can be added to the device (FIG. 8). The UVC disinfection module (8.6) contains at least one UV lamp in its structure. The UV lamp surface disinfection unit will start to disinfect the floor with UV-C light of 254 nm wavelength upon the command it receives from the joint electronic control and hardware unit (3).

The disinfection operation ends when the power supply (2) is low or the mapped space is completely treated, and the device returns to the filling station. While this unit is installed, the bottom cleaning of the device will not be activated at the filling station (A) in order not to damage the lamps.

If desired, the UVC disinfection module can be operated simultaneously with the cleaning module. During this operation, the UVC disinfection module (8.6) and/or the cleaning module can only be operated in certain parts of the cleaned environment. In this way, the possibility of harming the environment due to the non-process operation of the UVC disinfection module (8.6) will be eliminated.

According to the preferred embodiment of the invention, the UVC disinfection module (8.6) is positioned upper part of the robotic mechanism. According to FIG. 8, the UVC disinfection module (8.6) is connected to the upper connection port (17) formed in the upper part of the robotic mechanism.

The upper connection port (17) can also be used to connect other modules (8) to be operated by positioning them on the upper part of the robotic mechanism.

For example, the manual cleaning module (8.2) shown in FIG. 5 can be operated by connecting to the upper connection port (17) according to the preferred embodiment of the invention.

The robotic mechanism of the invention can also contain a negative ion generator module in order to clean the air of the cleaned environment in addition to floor disinfection.

While the device performs the cleaning or disinfection function, the negative ion generator module can also be used to increase the ambient air quality.

This unit can be added to the system also as a separate accessory module.

Another module that can be added to the robotic mechanism can be the chemical fogging module. The chemical fogging module aims to disinfect by fogging of the chemical substance.

Similarly, the chemical fogging module can be operated with other modules or separately. If desired, it can perform disinfection at the same time with cleaning or it can be operated only in certain parts of the place.

According to the preferred embodiment of the invention, the modular structure can be operated by connecting it to the device only when necessary. Again, according to this embodiment, it can be operated independently of the cleaning modules, by being attached to the robot.

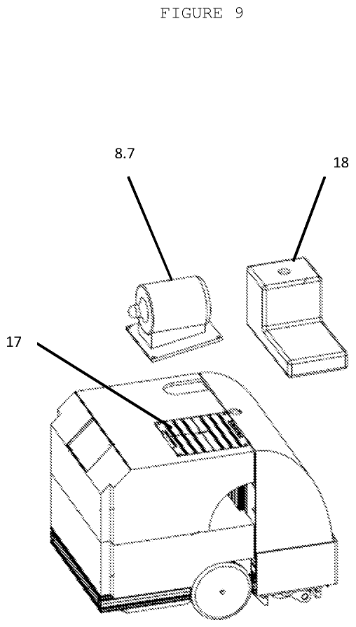

FIG. 9 shows the ULV (ultra low volume) disinfection module (8.7), which can be used for chemical fogging, together with the robotic mechanism, in demounted form.

According to one of the embodiments of the invention, the ULV disinfection module (8.7) can be operated by connecting it to the upper connection port (17).

According to FIG. 9, the ULV disinfection module (8.7) is operated connected to a ULV tank (18) structured in relation to the robotic mechanism.

The robotic mechanism described above with different modules (8) can be operated in a way that is joint to the main body design. It can be designed jointly in the main body design to accept all kinds of different modules (8) that can be controlled by the software of the joint electronic control and hardware unit (3) and recognize it when it is changed by the user. In this case, the device will be able to recognize each connected module (8) and will be able to directly operate the task of this module (8).

The joint main body (1) of the device is made of aluminium Σ(sigma) profiles in terms of both lightness and strength, ease of production and modularity. With the use of these profiles, it will be possible to intervene very easily in any malfunction, all kinds of design and hardware changes can be made quickly, and when necessary, additions and deductions can be made without completely spoiling the structural feature.

The device returns to the filling station (A) when the user-defined function is terminated, the power supply (2) is low, or the mapped space is completely treated. The filling station (A) is equipped with an energy input and the corresponding electronic structure that will enable the power supply (2) to be charged. The power supply (2) is designed as a unit that calculates the amount of charge remaining in the device and provides the most optimum filling. The filling station (A) can also be equipped with a clean water inlet with an electromagnet on-off valve and a liquid drainage port which can be remotely controlled by the device.

1. A mobile industrial autonomous device for operations such as polishing, mowing, fertilizing and spraying to be performed autonomously, which can;
move automatically with the lidar unit (4) and camera units on itself, scan and map the environment, determine a route accordingly and perform its function;
by using these maps stored in its memory and by means of the detectors (5) on it, define the obstructions/spatial changes and record these;
be operated connected to a filling station (A) to which it can return after completing its operation and clean and prepare itself for a new duty cycle, characterized in that;
it can comprise simultaneously or separately modules (8) of different features to be used to perform different functions and which can be changed on the same platform by the user by mounting and demounting.

2. A mobile industrial autonomous device according to claim 1, characterized in that it comprises modules (8) that can be connected to the device by humans.

3. A mobile industrial autonomous device according to claim 1, characterized in that it comprises modules (8) that can be mounted and demounted to the device autonomously without human interference in the filling station (A).

4. A mobile industrial autonomous device according to claim 1, characterized in that it comprises the joint main body (1) on which is a module slot (9) that allows the mounting-demounting of different modules (8) thereon.

5. A mobile industrial autonomous device according to claim 4, characterized in that it comprises the joint main body (1) which is also associated to at least one power supply (2) and to joint electronic control and hardware unit (3).

6. A mobile industrial autonomous device according to claim 5, characterized in that it comprises detectors (5) in the form of sensors consisting of one or more of IR impact sensor (5.1), US impact sensor (5.2), TOF sensor (5.3), IR fall sensor (5.4) and IR wall tracking sensor (5.5).

7. A mobile industrial autonomous device according to claim 5 or 6, characterized in that it comprises the joint main body (1) equipped with the bumper (6) associated with micro switches.

8. A mobile industrial autonomous device according to claim 1, characterized in that it comprises modules (8) that are associated with the filling-discharge units (7) for the liquid to be transferred to them.

9. A mobile industrial autonomous device according to claim 8, characterized in that it comprises modules (8) equipped with one or more of module liquid inlet (12), module energy input-output (10) and air suction connection (11).

10. A mobile industrial autonomous device according to claim 1, characterized in that it comprises the dry cleaning module (8.1).

11. A mobile industrial autonomous device according to claim 10, characterized in that it comprises the dust container structured within or associated with filling-discharge units (7).

12. A mobile industrial autonomous device according to claim 1, characterized in that it comprises the manual cleaning module (8.2) which allows the manual operation of the device along with the autonomous operation.

13. A mobile industrial autonomous device according to claim 12, characterized in that it comprises the manual cleaning module (8.2) which is located in the upper empty part of the device and which also contains the hose and the cleaning noses in an additional box.

14. A mobile industrial autonomous device according to claim 1, characterized in that it comprises the disc brush washing module (8.3) and/or cylinder brush washing module (8.4) for the robotic mechanism to perform the wet cleaning operation.

15. A mobile industrial autonomous device according to claim 14, characterized in that it comprises the dirty water tank (7.1) structured within or associated with the filling-discharge units (7) such as clean water tank and etc.

16. A mobile industrial autonomous device according to claim 1, characterized in that it comprises the polishing module (8.5) for the robotic mechanism to perform the polishing operation.

17. A mobile industrial autonomous device according to claim 16, characterized in that it comprises polish supply tank, the filling members of which are structured within or associated with the filling-discharge units (7).

18. A mobile industrial autonomous device according to claim 1, characterized in that it comprises the filling station (A) equipped with robotic washing unit (14).

19. A mobile industrial autonomous device according to claim 18, characterized in that it comprises the filling station (A) equipped with the pool structure created to drain the dirty water accumulating in the robotic washing unit (14).

20. A mobile industrial autonomous device according to claim 18, characterized in that it comprises the robotic washing unit (14) equipped with the moving comb system.

21. A mobile industrial autonomous device according to claim 1, characterized in that it comprises the filling station (A) equipped with charging unit (15).

22. A mobile industrial autonomous device according to claim 1, characterized in that it comprises the filling station (A) equipped with liquid filling inlet (16).

23. A mobile industrial autonomous device according to claim 1, characterized in that it comprises the HOCl preparation unit and hot water/steam preparation unit in association with the filling station (A).

24. A mobile industrial autonomous device according to claim 1, characterized in that it comprises spraying unit.

25. A mobile industrial autonomous device according to claim 1, characterized in that it comprises UVC disinfection module (8.6).

26. A mobile industrial autonomous device according to claim 1, characterized in that it comprises an upper connection port (17) located on the upper side of the robotic mechanism for the modules (8) to be connected.

27. A mobile industrial autonomous device according to claim 1, characterized in that it comprises the negative ion generator module.

28. A mobile industrial autonomous device according to claim 1, characterized in that it comprises the chemical fogging module.

29. A mobile industrial autonomous device according to claim 28, characterized in that it comprises ULV disinfection module (8.7) which can be operated by being connected to the upper connection port (17).

30. A mobile industrial autonomous device according to claim 29, characterized in that it comprises the ULV disinfection module (8.7) which can be operated in connection to a ULV tank (18) structured in association with the robotic mechanism.

31. A mobile industrial autonomous device according to claim 1 or 4, characterized in that it comprises the joint body (1) made of aluminium sigma profiles.
